(19) United States
(12) Patent Application Publication
(10) Pub. No.: US 2008/0097407 A1
(43) Pub. Date: Apr. 24, 2008

(54) LUER ACTIVATED DEVICE WITH COMPRESSIBLE VALVE ELEMENT

(76) Inventor: Michael Plishka, Lake Villa, IL (US)

Correspondence Address: BAXTER HEALTHCARE CORPORATION, 1 BAXTER PARKWAY, DF2-2E, DEERFIELD, IL, 60015, US

(21) Appl. No.: 11/550,570

(22) Filed: Oct. 18, 2006

(51) Int. Cl. A61M 25/00

(52) U.S. Cl. 604/533; 251/149; 604/246

(57) ABSTRACT

A luer activated device includes an inlet adapted to receive a male luer, an outlet associable with a fluid flow system, and a flow path defined therebetween. The inlet receives a resealable valve element having a aperture adapted to receive the male luer. The valve element is compressible, such that a male luer inserted into the aperture will cause the valve element to primarily compress, rather than stretching and deforming. One end of the aperture may include a pressure activated flow passage, which is adapted to open upon fluid flow through a male luer received by the aperture. Methods of using such luer activated devices are also provided.

### FIELD OF THE INVENTION

The present invention relates generally to luer activated devices or valves that allow for the bi-directional transfer of fluids to and from medical fluid flow systems.

### BACKGROUND OF THE INVENTION

Luer activated devices (LAD) or valves (LAV) are commonly used in association with medical fluid containers and medical fluid flow systems that are connected to patients or other subjects undergoing diagnostic, therapeutic or other medical procedures. A LAD can be attached to or part of a fluid container or a medical fluid flow system to simplify the addition of fluids to or withdrawal of fluids from the fluid flow system.

Within the medical field there are a wide variety of medical fluid flow systems, serving a variety of functions. One of the more common uses of LADs are in association with fluid flow systems that are used for the intravenous administration of fluids, such as saline, antibiotics, or any number of other medically-related fluids, to a patient. These flow systems are commonly referred to as intravenous or “IV” fluid administration sets, and use plastic tubing to connect a phlebotomized subject to one or more medical fluid sources, such as intravenous solution or medicament containers.

Typically, such intravenous administration sets include one or more LADs providing needless access to the fluid flow path to allow fluid to be added to or withdrawn from the IV tubing. The absence of a needle for injecting or withdrawing fluid has the important advantage of reducing the incidence of needle stick injuries to medical personnel. A LAD typically includes a tapered female luer component, such as the inlet into a valve housing, that accepts and mates with a tapered male luer of a medical infusion or aspiration device, such as a needleless syringe or a administration set tubing brand.

There are certain characteristics and qualities of LADs that are highly desirable. For example, the LAD should provide a sufficient microbial barrier for the full service life of the valve. It is desirable that the microbial barrier be conducive to the application of standard aseptic techniques preformed by clinicians during the use of the device. For example, the geometry of the LAD should be such that it is easily swabbable and reduces the potential of entrapping particulates or contaminants that cannot be cleanly swabbed clear prior to use.

Furthermore, it is highly desirable that the LAD be substantially devoid of any interstitial space or any other “dead space” that cannot be flushed, or that such interstitial space be physically isolated from the fluid flow path. Such interstitial space has the potential of providing an environment for undesired microbial growth. In addition, the LAD should have a geometry that allows it to be sufficiently flushed so as to clear the dynamic fluid path and adjacent areas of residual blood or intravenous fluids to prevent undesired clotting or microbial growth.

LAD's are commonly used with intravenous catheters that provide access to a patient's vascular system. In such systems, another desirable feature of a LAD is minimal displacement of fluid during insertion and removal of the male luer. In certain situations, it is preferable that the LAD be a neutral/neutral device in that there is zero or only a very slight displacement of fluid during both insertion and removal of the male luer. In other situations it can be desirable for the LAD to produce a positive displacement of fluid from the valve housing during the removal of the male luer. The LAD also preferably prevents blood reflux into the catheter. Reflux is known to reduce the efficiency of the catheter and contribute to catheter clotting.

In most situations it is preferred that the LAD be ergonomically dimensioned to be completely activated by a wide range of ISO compliant male luer lock adaptors. However, there may some instances when the LAD may be designed to be activated by a male luer connector that is not ISO complaint or is a male luer slip connector. Another desirable characteristic of a LAD is the ability of the LAD to seal against pressure contained within a fluid system to which the LAD is connected. For example, it is desirable to be leak resistance to positive pressures ranging from 10 to 45 psi and to negative pressures or vacuum from 1 to 5 psi. The LAD also preferably has a geometry that allows for easy priming and flushing that does not require any additional manipulations to remove residual air bubbles from the tubing system.

These and other desirable characteristics, which may be used separately or in combination, is preferably present over the full service life of the valve. When used in connection with an IV set or catheter, the LAD may go through many connections and disconnections. It is desirable that the life of an LAD last through upwards to about 100 connections and disconnections or 96 hours of dwell time.

As described more fully below, the fluid access devices of the present invention provides important advances in the safe and efficient administration or withdrawal of medical fluids to or from a fluid flow system.

### SUMMARY OF THE INVENTION

In accordance with one aspect of the present invention, a luer activated medical valve for the bi-directional transfer of fluid is provided with a valve housing having an inlet adapted to receive a male connector, preferably a male luer connector, an outlet, and a flow path defined therebetween. A valve element is fixedly received within the inlet of the valve housing and includes a resealable slit adapted to receive a male luer to allow fluid to be transferred between the male luer and the flow path. The valve element substantially comprised of a compressible material that substantially compresses to accommodate the male luer as at least a portion of the male luer is inserted into and through the slit.

According to another aspect of the present invention, a medical valve for the bi-directional transfer of fluid is provided with a valve housing having an inlet adapted to receive a male luer, an outlet and a flow path defined therebetween. A valve element is fixedly received within the inlet of the valve housing and substantially comprised of a compressible material. The valve element includes a resealable slit adapted to receive at least a portion of a male luer. One end of the slit includes at least one fluid flow passage and the flow passage is adapted to open upon fluid flow through a male luer at least partially received by the slit.

According to yet another aspect of the present invention, a method of transferring fluid between a male luer and a fluid flow system involves providing a medical valve having a sealed inlet adapted to receive a male luer, an outlet in fluid communication with a fluid flow system, and a flow path defined therebetween. A male luer is inserted into the inlet and through a resealable slit of a valve element fixedly received within the inlet. So inserting the male luer compresses the valve element to allow fluid communication between the male luer and the flow path of the medical valve. When the male luer has compressed the valve element, fluid flow is commenced through one of the fluid flow system and the male luer and into the flow path of the medical valve

### BRIEF DESCRIPTION OF THE DRAWINGS

Turning now to a more detailed description of the various embodiments of the present invention illustrated in the attached drawings, of which:

FIG. 1 is a cross-sectional view of one embodiment of a luer activated device of the present invention;

FIG. 2 is a cross-sectional view of the LAD of FIG. 1, shown with a male luer inserted thereinto for fluid transfer;

FIG. 3 is a cross-sectional view of another embodiment of an LAD according to the present invention employing a vent passageway;

FIG. 4 is a cross-sectional view of the LAD of FIG. 3, shown with a male luer inserted thereinto for fluid transfer;

FIG. 5 is a cross-sectional view of another embodiment of an LAD according to the present invention;

FIG. 6 is a cross-sectional view of the LAD of FIG. 5, shown connected with a male luer inserted thereinto for fluid transfer; and

FIG. 7 is a cross-sectional view of the LAD of FIG. 6, shown with fluid flow through the male luer.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but as a representative basis for teaching one skilled in the art to variously employ the present invention in virtually any appropriate manner.

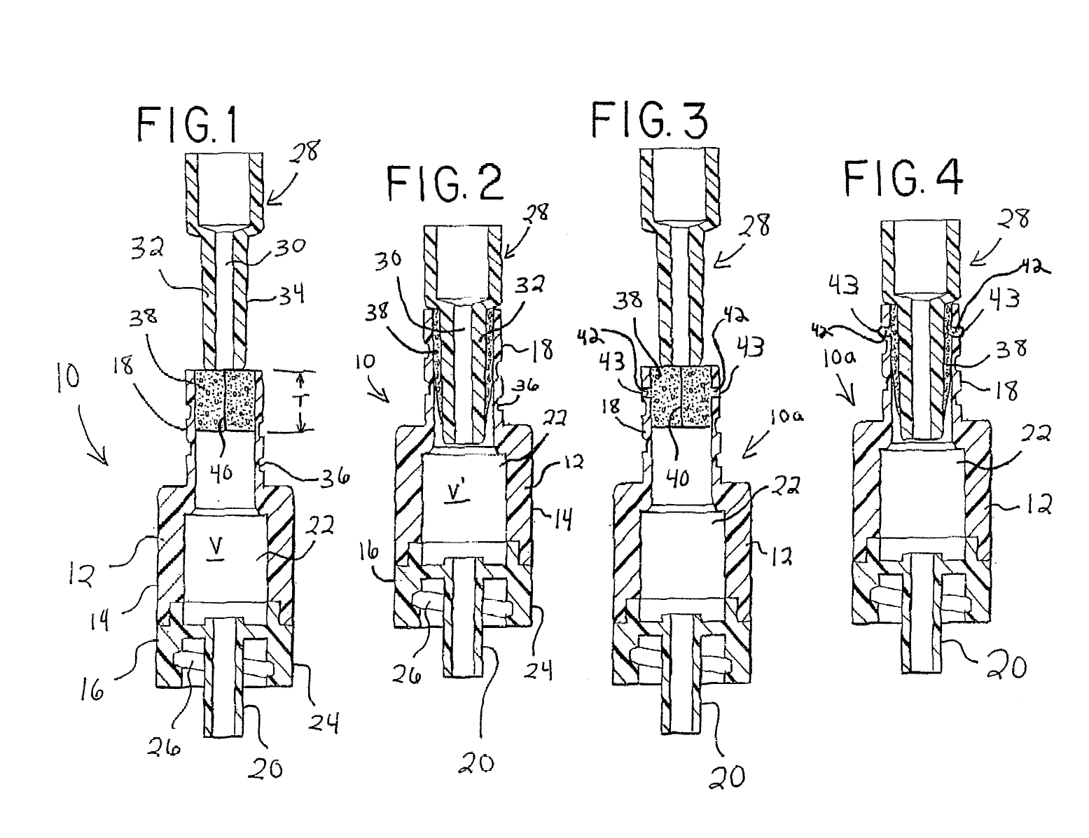

FIGS. 1 and 2 generally illustrate a first embodiment of a luer activated device (LAD) or valve of the present invention, generally designated as 10. The LAD 10 includes a valve housing 12 preferably comprised of a rigid material, such as rigid plastic or other suitable material. The LAD 10 may be provided as a unitary structure (not illustrated) or as a combination of a joined upper housing portion 14 and a lower housing portion 16. The LAD 10 also includes an inlet 18, an outlet 20, and a flow path 22 defined therebetween. The terms “inlet” and “outlet” are not to be interpreted as limiting the LAD 10 to applications involving fluid flow in a particular direction, e.g., from the inlet 18 to the outlet 20, because LAD's according to the present invention may be used in applications involving fluid flow from the inlet 18 to the outlet 20 or from the outlet 20 to the inlet 18.

The outlet 20 is adapted to be connected to any of a number of fluid flow systems, so the exact configuration of the outlet 20 will vary according to the nature of the fluid flow system to which it is to be connected. For example, the illustrated outlet 20 is suitable for use in connecting the valve 10 to an IV administrative tubing set (not illustrated). In the embodiment of FIGS. 1 and 2, the outlet 20 includes a skirt 24 defining an internal thread 26, which may be adapted to engage an external thread of the associated fluid flow system (not illustrated). Of course, the outlet 20 may be provided with a different configuration, a different locking system, or without a locking system, depending on the anticipated usage of the valve 10. Also, the valve may be formed as an integral part of a larger structure without departing from the present invention.

The inlet 18 is adapted to receive a male connector such as a luer 28 according to known structure and operation. The inlet 18 and male luer 28 preferably conform to ISO and/or ANSI standards. The typical male luer 28 has a hollow channel 30 defined by a generally tubular wall 32. The wall 32 preferably has a substantially smooth outer surface 34 which is typically slightly tapered. The inlet 18 may include external threads 36 (FIGS. 1 and 2), in which case a portion of the luer wall 32 may be surrounded by a collar member (not illustrated) having internal threads adapted to removably lock the male luer 28 to the inlet 18. Other locking mechanisms may also be incorporated into LAD's according to the present invention.

To control flow through the housing 12, a deformable septum or valve element 38 having an aperture 40 (preferably but not exclusively in the form of a slit) therethrough is fixedly mounted to normally block and seal the inlet 18. The valve element 38 acts as a microbial barrier between the internal fluid flow path 22 of the LAD 10 and the atmosphere. The valve element 38 may be fixedly attached to the inlet 18 by any of a number of means. Suitable means include, but are not limited to, adhesive or mechanical bonding and interference overmolding. Preferably, the valve element 38 is slightly larger than the inlet 18, such that it is radially compressed to some extent in the closed condition of FIG. 1. Imparting such compression to the valve element 38 promotes an improved seal of the resealable valve element slit 40, thereby preventing fluid leakage through the inlet 18. At the inlet, the valve element 38 preferably has a substantially flat or slightly outwardly curved outside surface that can be easily wiped with antiseptic, which aids in presenting contamination during use.

The valve element slit 40 is adapted to accept the male luer 28 and allow the male luer 28 to access the interior of the LAD 10. The slit 40 may be integrally formed, e.g., molded, with the valve element 38 or may be formed after the valve element 38 is manufactured or seated within the inlet 18 such as by a slitting operation.

In a closed (FIG. 1) the valve element 38 assumes a substantially cylindrical shape to close the slit 40 and prevent fluid flow through the inlet 18. In an open (FIG. 2), the valve element 38 is forced into a deformed, tubular shape by the male luer 28 received by the slit 40. The radius of the inlet 18 is greater than the radius of the male luer 28, and the deformed valve element 38 of FIG. 2 occupies and seals the space therebetween to prevent fluid leakage from the inlet 18.

The valve element 38 is substantially comprised of a deformable, compressible material. When used herein, the term “compressible” refers to a material that is capable of decreasing in volume by more than a nominal amount upon insertion of a male luer 28 into the inlet 18 (FIG. 2). For example, a silicone or elastomeric slit valve element according to known structure and operation is deformable, because it will change shape to accommodate a male luer, but it is not compressible because it is not capable of a substantial reduction in volume. Those of ordinary skill in the art will appreciate that, when using known elastomeric slit septa, the open internal volume of the valve (i.e., the portion of the valve interior that is available for fluid flow) will substantially decrease upon insertion of a male luer, because the valve interior must receive the combined volumes of the male luer and the deformed valve element, instead of just the volume of the valve element. This change in open internal volume may impart a positive displacement of fluid through the outlet during the insertion of the male luer, which may be undesirable in certain applications.

Through the use of a compressible valve element 38, the change in available flow path volume from V (FIG. 1) to V′ (FIG. 2) may be reduced or minimized to limit or avoid the effects of positive fluid displacement. The pre-insertion open internal volume V is equal to the volume of the housing interior less the volume of the closed valve element 38 (FIG. 1), while the post-insertion open internal volume V′ is equal to the volume of the housing interior less the combined volume of the portion of the male luer 28 received with in the housing 12 (illustrated in FIG. 2 as the entire tubular luer wall 32) and the volume of the deformed valve element 38. From the foregoing relationship, it will be seen that the volume of the closed valve element 38 (FIG. 1) is preferably equal to the sum of the volumes of the deformed valve element 38 and the portion of the male luer 28 received within the LAD housing 12 (FIG. 2) to eliminate any change in open internal volume before and after insertion of the male luer 28. This relationship may be manipulated by changing any of a number of factors, including the size of the luer portion received by the inlet 18, the difference in radii between the inlet 18 and the luer wall 32, and the thickness T of the valve element 38.

Another benefit of using a compressible material instead of a solely deformable material is that the valve element 38 is subjected to less shear stress upon insertion of the male luer 28 and tends to be more durable. In particular, it will be appreciated by those of ordinary skill in the art that a typical rubber or silicone slit valve element is significantly stretched and deformed upon insertion of a male luer, which puts the material primarily in a state of shear stress. In contrast, septa according to the present invention are primarily radially compressed by the male luer 28, with a smaller degree of deformation and shear stress. Accordingly, the majority of the stress is transmitted to the bonding material between the valve element 38 and the inlet 18, which is significantly stronger in compression than a rubber or silicone valve element is in shear, so the valve element 38 may be more durable than known rubber or silicone septa.

Preferably, the valve element 38 is substantially comprised of a compressible polymeric foam, such as a silicone or urethane foam. The foam may be provided with a closed- or open-cell structure, depending on the intended use of the LAD 10. A closed-cell structure is typically more rigid and less compressible than an open-cell structure, so such a configuration may be preferred for application requiring less deformation of the valve element 38, such as when the valve 10 is used in combination with a male luer 28 having a relatively small radius.

Open-cell foams may be used in applications requiring more deformation, such as when the valve 10 is used in combination with a male luer 28 having a relatively large radius. Open-cell foams also allow for other variations that are not possible or not as practicable with closed-cell foams. For example, an open-cell foam may be impregnated with a liquid or gelatinous material having anti-microbial, anti-clotting, lubricating, or other properties. When the male luer 28 is inserted into the slit 40, the valve element 38 is compressed, thereby applying the material to the male luer 28, the flow path 22, or the fluid being transferred through the valve 10.

While open-cell foams are more versatile in certain respects than closed-cell foams, there is the risk that the open cells may allow fluid leakage through the inlet 18, especially in the uncompressed condition of FIG. 1. To prevent such leakage, an open-cell foam may be treated with a substantially closed outer layer or skin (not illustrated), which may be applied by any of a number of methods, including dipping. Preferably, such a skin is sufficiently porous to allow elution of a material impregnated within the foam, without allowing fluid leakage through the inlet 18. Suitable skin materials include silicon, ePTFE or urethane While such surface treatment is more preferred for use with open-cell foams, a skin or outer layer may also be applied to a closed-cell foam (not illustrated). The skin may have different characteristics than the underlying valve element 38, to make it easier to insert or remove the male luer 28, for example.

FIGS. 3 and 4 illustrate another embodiment of a valve 10a suitable for use with an open-cell foam. The embodiment of FIGS. 3 and 4 conforms to the foregoing description of the embodiment of FIGS. 1 and 2, except that the inlet 18 includes at least one vent 42. Each vent 42 defines a lateral opening or aperture through the wall of the housing at inlet 18 that allows for communication between the valve element 38 and the ambient atmosphere. In the uncompressed condition of FIG. 3, e.g., air is maintained within the open cells of a foam valve element 38. When the male luer 28 is at least partially inserted into the slit 40 of the valve element 38 (FIG. 4), the open cells are compressed and the air maintained therein is vented to the atmosphere through the vents 42. If the valve element 38 is provided with a skin or outer layer, then the portion adjacent to the vents 42 is preferably uncoated to promote aspiration of the foam.

In an alternate embodiment the vent 42 may be sized to allow for a portion of the valve element 38 to be move from the interior of the housing during compression of the valve element by the luer connector giving more volume into which the valve element may be displaced. In a further embodiment a resilient membrane 43 extends around the exterior of the inlet 18 and is sealed to the housing 12 along its upper and lower edge so that it covers the vents 42. When air is vented through the vents 42 the air is captured by the membrane 43. When the luer connector is removed the membrane 43 forces the air to reenter the inlet through the vents 42 which facilitates recovery of the valve element 38.

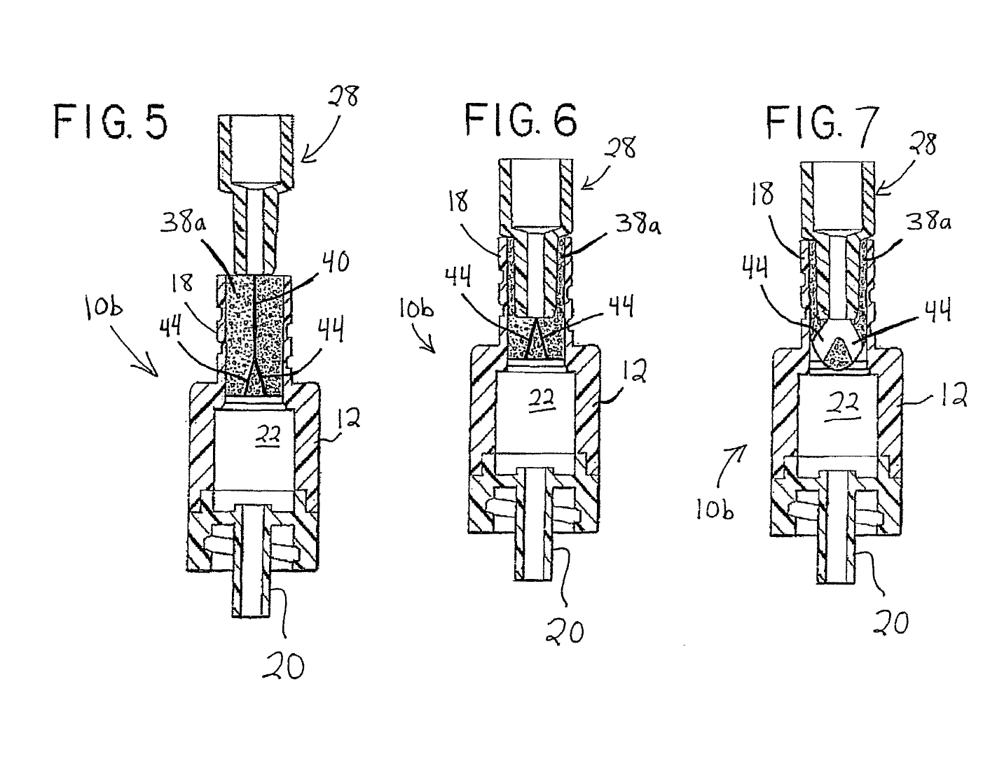

FIGS. 5-7 illustrate a valve 10b according to another aspect of the present invention. FIGS. 5-7 conform to the foregoing description of the embodiment of FIGS. 1 and 2, except for the structure and operation of the valve element. The valve element 38a illustrated in FIGS. 5-7 is thicker in axial dimension than the valve element 38 illustrated in FIGS. 1 and 2, and includes at least one pressure-activated flow passage 44 at a lower end of the slit 40. The valve element 38a is adapted such that full insertion of the male luer 28 will substantially open the slit 40, but not the pressure-activated flow passages 44 (FIG. 6).

In the position of FIG. 6, fluid flow through the inlet 18 is prevented by the closed pressure-activated flow passages 44. The pressure-activated flow passages 44 remain closed until fluid flow through the male luer 28 is commenced (FIG. 7), at which time the resulting pressure increase causes the pressure-activated flow passages 44 to open and allow flow through the LAD 10b. When fluid flow through the male luer 28 ceases, the pressure-activated flow passages 44 automatically close to prevent further flow through the valve 10b (FIG. 6). Hence, it will be seen that the flow passages 44 act to prevent or limit the valve from drawing fluid into the outlet as the male luer is withdrawn from the position in FIG. 6 to the position in FIG. 5, and also act as an auxiliary seal to prevent fluid leakage through the inlet 18 when the male luer 28 is inserted into the valve element slit 40.

In an alternate embodiment, the flow passages 44 are opened when the male luer 28 is inserted primarily by being stretched open by the resilient restraint of the valve element 38 by the housing. Upon the withdrawal of the male luer 28, the stretching is reduced and the flow passages 44 close.

Preferably, the flow passages 44 are adapted to provide a positive fluid displacement upon closing. In other words, upon moving from the open condition of FIG. 7 to the closed condition of FIG. 6, the flow passages 44 preferably force fluid contained therein toward the valve outlet 20, rather than toward the inlet 18 and male luer 28.

Individual aspects of the various embodiments may be combined without departing from the scope of the present invention. For example, the valve element 38a of FIGS. 5-7 may be provided with a lateral vent 42 (FIGS. 3 and 4) or may be adapted to elute a liquid or gelatinous material when compressed.

While the present invention has been described in terms of certain preferred and alternative embodiments for purposes of illustration, it is not limited to the precise embodiments shown or to the particular features, shapes or sizes illustrated. A variety of changes may be made without departing from the present invention as defined by the appended claims.

1. A medical valve for the bi-directional transfer of fluid, comprising:
a valve housing having an inlet adapted to receive a male luer, an outlet and a flow path defined therebetween; and
a valve element fixedly received within the inlet of the valve housing, said valve element including a resealable slit adapted to receive a male luer to allow fluid to be transferred between the male luer and the flow path, said valve element substantially comprised of a compressible material that substantially compresses to accommodate the male luer as at least a portion of the male luer is inserted into and through the operative of the valve element, wherein there is substantially no fluid displacement when the male luer is inserted or removed from the aperture.

2. The medical valve of claim 1 in which the compressible material is a polymeric foam.

3. The medical valve of claim 2 in which the polymeric foam is a silicone or urethane foam.

4. The medical valve of claim 2 in which the polymeric foam is a closed-celled foam.

5. The medical valve of claim 2 in which the polymeric foam is an open-celled foam.

6. The medical valve of claim 5, further comprising at least one vent in the valve housing, which vent is adapted to transfer air from the valve element to the ambient atmosphere when the valve element is compressed.

7. The medical valve of claim 1, wherein the valve element further comprises an antiseptic accent therein releasable upon compression.

8. The medical valve of claim 1, wherein the volume of the valve element in an uncompressed condition is substantially the same as the combined volumes of the valve element in a compressed condition and the portion of a male luer received within the inlet when the male luer is fully inserted into the inlet.

9. A medical valve for the bi-directional transfer of fluid, comprising:
a valve housing having an inlet adapted to receive a male luer, an outlet and a flow path defined therebetween; and
a valve element fixedly received within the inlet of the valve housing and substantially comprised of a compressible material, said valve element including
a resealable aperture adapted to receive at least a portion of a male luer and
at least one fluid flow passage at an end of the aperture, wherein said flow passage is adapted to open upon fluid flow through a male luer at least partially received by the slit.

10. The medical valve of claim 9 in which the material of the valve element is a polymeric foam.

11. The medical valve of claim 10 in which the polymeric foam is a silicone or urethane foam.

12. The medical valve of claim 10 in which the polymeric foam is a closed-celled foam.

13. The medical valve of claim 10 in which the polymeric foam is an open-celled foam.

14. The medical valve of claim 13, further comprising at least one vent in the valve housing, wherein the vent is adapted to transfer air from the valve element to the ambient atmosphere when the valve element is compressed.

15. The medical valve of claim 9, wherein said fluid flow passageway is adapted to automatically close upon cessation of fluid flow through a male luer at least partially received by the valve element aperture.

16. The medical valve of claim 9, wherein the valve element further comprises an antiseptic agent therein releasable upon compression.

17. A method of transferring fluid between a male luer and a fluid flow system, comprising:
providing a medical valve having a sealed inlet adapted to receive a male luer, an outlet in fluid communication with a fluid flow system, and a flow path defined therebetween;
inserting a male luer into the inlet and through a resealable aperture of a compressible valve element fixedly received within the valve, thereby compressing the valve element to allow fluid communication between the male luer and said flow path of the medical valve, wherein there is substantially no fluid displacement when the male luer is inserted into the aperture; and
commencing fluid flow through one of the fluid flow system and the male luer and into the flow path of the medical valve.

18. The method of claim 17, wherein said providing a medical valve includes providing a valve element substantially comprised of a polymeric foam.

19. The method of claim 17, wherein said providing a medical valve includes providing a valve element substantially comprised of a silicone or urethane foam.

20. The method of claim 17, wherein said providing a medical valve includes providing a valve element substantially comprised of a closed-celled foam.
